(19) United States
(12) Patent Application Publication
(10) Pub. No.: US 2015/0038404 A1
(43) Pub. Date: Feb. 5, 2015

(54) MACROCYCLIC COMPOUNDS

(71) Applicant: University of Florida Research Foundation, Gainesville, FL (US)

(72) Inventors: Hendrik Luesch, Gainesville, FL (US); Valerie J. Paul, Fort Pierce, FL (US); Lilibeth A. Salvador, Gainesville, FL (US)

(21) Appl. No.: 14/381,168

(22) PCT Filed: Feb. 22, 2013
(86) PCT No.: PCT/US2013/027384

§ 371 Date: Aug. 26, 2014

Related U.S. Application Data
(60) Provisional application No. 61/603,617, filed on Feb. 27, 2012.

(51) Int. Cl. C07K 7/64; A61K 38/12

(52) U.S. Cl. CPC C07K 7/64; A61K 38/12
USPC 514/1.8; 530/321; 514/20.5; 514/19.3; 514/4.8

(57) ABSTRACT

This invention relates to novel macrocyclic compounds (e.g., those delineated in the formulae herein), pharmaceutically acceptable salts, solvates, and hydrates thereof. This invention also provides compositions comprising a compound of this invention and the use of such compounds and compositions in methods of treating diseases and conditions that are beneficially treated by administering inhibitors of the protease elastase.

### CROSS-REFERENCE TO RELATED APPLICATION

This application claims the benefit of U.S. Provisional Application No. 61/603,617, filed on Feb. 27, 2012, the entire contents of which are incorporated herein by reference.

### STATEMENT OF RIGHTS TO INVENTIONS MADE UNDER FEDERALLY SPONSORED RESEARCH

This work was supported in part by National Institutes of Health, Grant No. NIGMS P41GM086210. The government has certain rights in the invention.

### BACKGROUND OF THE INVENTION

Many dolastatin analogs isolated from marine cyanobacteria are termed lyngbyastatins and symplostatins, isolated from Lyngbya or Symploca spp., respectively. Many of them have been shown to be protease inhibitors, all of which are analogs of dolastatin 13.¹ Several non-cytotoxic marine cyanobacterial metabolites have been demonstrated to be potent inhibitors of serine proteases, including elastase, chymotrypsin and trypsin. Lyngbyastatins 4-10 and the related compounds somamide B, kempopeptins A and B are cyclic depsipeptides isolated from different collections of Lyngbya sp. demonstrated to inhibit serine proteases.²⁻⁷ The backbone structure of these compounds is reminiscent of the sea hare metabolite dolastatin 13⁷ and the Symploca sp.-derived symplostatin 2.⁸ These compounds have high structural similarity in the core depsipeptide ring, bearing a modified Glu residue, 3-amino-6-hydroxy-2-piperidone (Ahp), which is suggested to confer the serine protease inhibitory activity.^{3, 4} The proteolytic activities of elastase, chymotrypsin, and trypsin were inhibited in varying degrees by these cyanobacterial metabolites, with lyngbyastatins and somamide B being potent elastase inhibitors, while kempopeptins A and B inhibited chymotrypsin and trypsin, respectively.³⁻⁵ Structure-activity relationships indicated that the residue between the Ahp unit and Val imparts selectivity towards elastase, chymotrypsin or trypsin. Having the modified Ser residue, 2-aminobutenoic acid (Abu) at this position, as present in dolastatin 13, symplostatin 2 and lyngbyastatin 4-10, confers selectivity for elastase while a basic amino acid such as Lys/Arg provides trypsin selectivity, and a nonpolar residue such as Leu gives both elastase and chymotrypsin selectivity. Elastase has been implicated in several disease states such as chronic obstructive pulmonary disease (COPD), cystic fibrosis and cancer.⁹⁻¹² It has been linked to inflammation via upregulation of IL-8.⁹ Its role in tumor invasion and metastasis was correlated to its ability to degrade extracellular matrix components.^{11, 12} The downstream cellular consequences of elastase inhibition by these potent cyanobacteria-derived metabolites have, however, not been demonstrated. Here we describe new elastase inhibitors structurally related to lyngbyastatins 4-10 and symplostatin 2 and characterize their structures and biological activities. Despite the beneficial activities of known inhibitors of the protease elastase, there is a continuing need for new compounds to treat the aforementioned diseases and conditions.

### SUMMARY OF THE INVENTION

This invention relates to novel macrocyclic compounds (e.g., those delineated herein), pharmaceutically acceptable salts, solvates, stereoisomers, esters, amides, and hydrates thereof. This invention also provides compositions comprising a compound of this invention and the use of such compounds and compositions in methods of treating diseases and conditions that are beneficially treated by administering inhibitors of the protease elastase.

In one aspect, the invention provides an isolated and/or purified compound of formula (I):

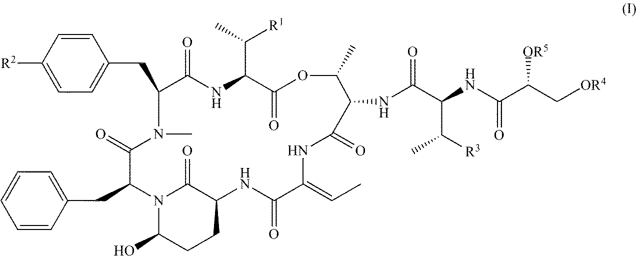

wherein:

R¹ is alkyl;

R² is H or hydroxyl;

R³ is alkyl or hydroxyl;

R⁴ is H or SO₃M⁺, wherein M⁺ is Na, K, or Li; and

R⁵ is H or alkyl;

or a pharmaceutically acceptable salt, ester, amide, hydrate, stereoisomer, or solvate thereof.

In another aspect, the invention provides an isolated and/or purified compound of formula (I):

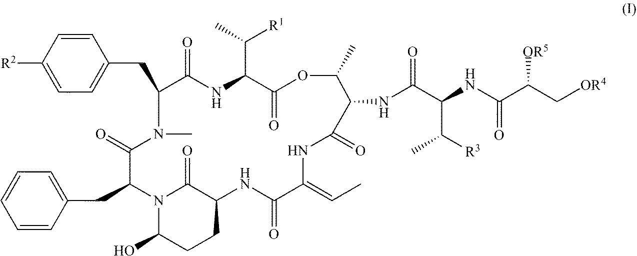

wherein:

R¹ is alkyl;

R² is H or hydroxyl;

R³ is alkyl or hydroxyl;

R⁴ is H or SO₃M⁺, wherein M⁺ is Na, K, H, or Li; and

R⁵ is H or alkyl;

with the proviso that when R¹ is alkyl, R² is H, R³ is alkyl, and R⁵ is alkyl,

then R⁴ is not H;

or a pharmaceutically acceptable salt, ester, amide, hydrate, stereoisomer, or solvate thereof.

In another embodiment, the compounds are those of formula (I), wherein R⁴ is SO₃Na.

In another embodiment, the compounds are those of formula (I), wherein R⁴ is SO₃Na; R¹ is independently alkyl; and R³ is independently alkyl.

In another embodiment, the compounds are those of formula (I), wherein R⁴ is SO₃M⁺.

In another aspect, the invention provides a pharmaceutical composition comprising a compound of any of the formulae delineated herein, or a pharmaceutically acceptable salt, ester, amide, hydrate, stereoisomer, or solvate thereof, together with a pharmaceutically acceptable carrier or diluent.

The invention also provides the use of a compound of any of the formulae herein, or a pharmaceutically acceptable salt, ester, amide, hydrate, stereoisomer, or solvate thereof, for the manufacture of a medicament for treatment of a disease or condition identified herein.

In another aspect, the invention provides a method of treating a protease elastase related disease or disorder in a subject (e.g., a subject identified as in need of such treatment), wherein the method comprises administering to the subject an effective amount of a compound of any of the formulae herein, or a pharmaceutically acceptable salt, ester, amide, hydrate, stereoisomer, or solvate thereof. In certain embodiments, the protease elastase related disease or disorder is chronic obstructive pulmonary disease (COPD), cystic fibrosis, cancer, lung tissue injury (emphysema), asthma, rheumatoid arthritis. In certain embodiments, the disease or disorder is allergic inflammation, artherosclerosis, or autoimmune diseases. In certain embodiments, the compounds and compositions herein are useful in providing and/or enhancing anti-aging properties of skin by preventing (e.g., UVA-induced) wrinkle formation.

In another aspect, the invention provides a method of treating a disease or disorder in a subject (e.g., a subject identified as being in need of such treatment), wherein the method comprises administering to the subject an effective amount of a compound of any of the formulae herein, or a pharmaceutically acceptable salt, ester, amide, hydrate, stereoisomer, or solvate thereof, or composition thereof. In certain embodiments, the disease or disorder is selected from the group consisting of chronic obstructive pulmonary disease (COPD), cystic fibrosis, cancer, lung tissue injury (emphysema), asthma, rheumatoid arthritis. In certain embodiments, the disease or disorder is allergic inflammation, artherosclerosis, or autoimmune diseases. In certain embodiments, the methods are useful in providing and/or enhancing anti-aging properties of skin by preventing (e.g., UVA-induced) wrinkle formation.

In another aspect, the invention provides a method of inhibiting elastase activity in a subject (e.g., a subject identified as being in need of such treatment), wherein the method comprises administering to the subject an effective amount of a compound of any of the formulae herein, or a pharmaceutically acceptable salt, ester, amide, hydrate, stereoisomer, or solvate thereof, or composition thereof.

In another aspect, the invention provides a method of reducing soluble ICAM-1 production in a subject (e.g., a subject identified as being in need of such treatment), wherein the method comprises administering to the subject an effective amount of a compound of any of the formulae herein, or a pharmaceutically acceptable salt, ester, amide, hydrate, stereoisomer, or solvate thereof, or composition thereof.

In another aspect, the invention provides a method of inhibiting a protease elastase in vitro, in a subject, or in a cell. The method comprising contacting a compound of any of the formulae herein or composition thereof with the cell and/or subject.

The invention also provides methods for isolation, structure determination, and biological determination of a compound of any of the formulae herein, or a pharmaceutically acceptable salt, ester, amide, hydrate, stereoisomer, or solvate thereof. The methods comprise one or a combination of steps or actions essentially as delineated herein, including those specifically recited in the examples herein.

### BRIEF DESCRIPTION OF THE DRAWINGS

The present invention is further described below with reference to the following non-limiting examples and with reference to the following figures, in which:

FIG. 1. Structures of symplostatins 5-10 (1-6), dolastatin 13, and lyngbyastatin 7.

FIG. 2. Lyngbyastatin 7 was cocrystallized with porcine pancreatic elastase with a resolution of 1.5 Å. A. Enzyme-inhibitor interactions are indicated, with the Abu moiety of lyngbyastatin 7 being the key residue for selective and potent elastase inhibition. B. The Abu moiety occupies the S1 subsite in the elastase catalytic site. The pendant side chain of lyngbyastatin 7 occupies other subsites of the enzyme.

FIG. 3. Cell viability of BEAS-2B cells treated with human neutrophil elastase (HNE, 100 nM) in the presence of DMSO or varying concentrations of symplostatin 5 (1) were evaluated using the 3-(4,5-dimethylthiazol-2-yl)-2,5-diphenyltetrazolium (MTT) assay. HNE induced cell death after 24 h and this was alleviated by cotreatment with compound 1.

FIG. 4. Human neutrophil elastase induces changes in morphology of BEAS-2B cells within 2 h of treatment. This effect was reverted by the elastase inhibitors symplostatin 5 (1) and sivelestat.

FIG. 5. A. Protein levels of sICAM-1 in culture supernatants treated with human neutrophil elastase was upregulated, in comparison to control. Significant decrease, in sICAM-1 levels were observed with treatment with >100 nM of symplostatin 5 (1) or 320 nM of sivelestat (P<0.05). B. In accordance, low levels of mICAM-1 were observed in whole cell lysates of elastase-treated cells, suggesting sICAM-1 production via the proteolytic cleavage of mICAM-1 by elastase. This is further corroborated by significant increase in mICAM-1 levels in symplostatin 5 (1) cotreated cells.

### DETAILED DESCRIPTION OF THE INVENTION

The invention features compounds, compositions, and methods of using such compounds for treating diseases or disorders. In certain embodiments, the compounds are novel bioactive compounds that are discovered from the source of marine cyanobacteria. In certain embodiments, the compounds of the invention are natural products and their analogs thereof. In one embodiment, the compounds are useful as elastase inhibitors.

The invention is based, at least in part, on the discovery of macrocyclic compounds referred to as symplostatins, including symplostatin 5 (1), symplostatin 6 (2), symplostatin 7 (3), symplostatin 8 (4), symplostatin 9 (5), and symplostatin 10 (6), through exploration of marine cyanobacteria as a source of novel bioactive compounds. The structures of the symplostatins were determined using NMR, MS, and chiral HPLC techniques. It was found surprisingly that symplostatins 5-10 (1-6) are capable of selectively inhibiting various proteases (such as, elastase).

### DEFINITIONS

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

As used in the specification and claims, the singular term “a”, “an” and “the” include plural references unless the context clearly dictates otherwise. For example, the term “a cell” includes a plurality of cells, including mixtures thereof.

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The term “agent” is meant a small molecule compound, a polypeptide, polynucleotide, or fragment, or analog thereof, or other biologically active molecule.

The term “chiral” refers to molecules which have the property of non-superimposability of the mirror image partner, while the term “achiral” refers to molecules which are superimposable on their mirror image partner.

The terms “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

With respect to the nomenclature of a chiral center, terms “d” and “I” configuration are as defined by the IUPAC Recommendations. As to the use of the terms, diastereomer, racemate, epimer and enantiomer is used in their normal context to describe the stereochemistry of preparations.

The term “effective amount” includes an amount effective, at dosages and for periods of time necessary, to achieve the desired result, e.g., sufficient to treat a disease or disorder delineated herein. An effective amount of compound of the invention may vary according to factors such as the disease state, age, and weight of the subject, and the ability of the compound of the invention to elicit a desired response in a cell or in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound (i.e., an effective dosage) may range from about 0.005 μg/kg to about 200 mg/kg, about 0.1 μg/kg to about 100 mg/kg, or about 1 mg/kg to about 50 mg/kg of body weight. In other embodiments, a therapeutically effect concentration may range from about 1.0 nM to about 1 μm. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 μg/kg to about 200 mg/kg of body weight, one time per day for between about 1 to 10 weeks, between 2 to 10 weeks, between about 1 to 8 weeks, or for about 4, 5, or 6 weeks. It will also be appreciated that the effective dosage of a compound used for treatment may increase or decrease over the course of a particular treatment.

The term “in combination with” is intended to refer to all forms of administration that provide an a compound of the invention together with an additional pharmaceutical agent, such as a second compound used in clinic for treating or preventing osteoclast-related disease or disorder, where the two are administered concurrently or sequentially in any order.

The term “compound,” as used herein, is also intended to include any salts, solvates or hydrates thereof.

The term “hydrate” means a compound of the present invention or a salt thereof, which further includes a stoichiometric or non-stoichiometric amount of water bound by non-covalent intermolecular forces.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The terms “isolated,” “purified,” “pure” or “biologically pure” refer to material that is substantially or essentially free from components (such as proteins, nucleic acids, carbohydrates, and other cellular materials) that normally accompany it as found in its native or natural state, e.g., its state in an organism in which the compound or material naturally occurs. Purity and homogeneity are typically determined using analytical chemistry techniques such as polyacrylamide gel electrophoresis or high performance liquid chromatography. In certain embodiments, a compound of this invention is at least 50% pure, 60% pure, 75% pure, 80% pure, 85% pure, at least 90% pure, or at least 95% pure (e.g., by weight). In certain instances, the compound is at least 98% pure, 99% pure, 99.5% pure, 99.8% pure, or 99.9% pure.

The term “modulate” refers to an increase or decrease, e.g., in the ability of a compound inhibiting activity of a target in response to exposure to a compound of the invention, including for example in an subject (e.g., animal, human) such that a desired end result is achieved, e.g., a therapeutic result.

The term “obtaining” as in “obtaining a compound” capable of modulating (agonizing, antagonizing) a target delineated herein includes purchasing, synthesizing or otherwise acquiring the compound.

The term “subject” includes organisms which are capable of suffering from a disorder as described herein or who could otherwise benefit from the administration of a compound of the present invention, such as human and non-human animals. Preferred humans include human patients suffering from or prone to suffering from diseases or disorders as discussed above, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc. A “subject identified as being in need of treatment” includes a subject diagnosed, e.g., by a medical or veterinary professional, as suffering from or susceptible to a disease, disorder or condition described herein.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylene sulfonate, phenylacetate, phenylpropionate, phenylbutyrate, citrate, lactate, i-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention will include both racemic mixtures, and also individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are well known in the art and may be applied as practicable to final compounds or to starting material or intermediates.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl.

“Stereoisomer” refers to both enantiomers and diastereomers.

“Tert”, “^{t}”, and “t-” each refer to tertiary.

“US” refers to the United States of America.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

Therapeutic Compounds

The present invention provides a compound of Formula I:

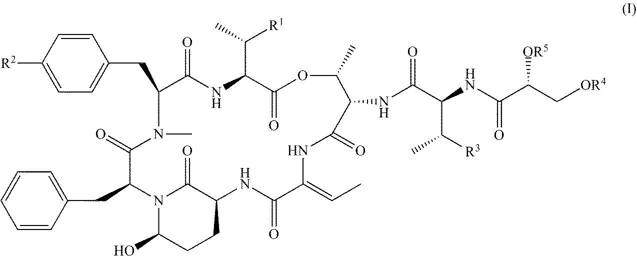

wherein:

R¹ is alkyl;

R² is H or hydroxyl;

R³ is alkyl or hydroxyl;

R⁴ is H or SO₃M⁺, wherein M⁺ is Na, K, or Li; and

R⁵ is H or alkyl;

or a pharmaceutically acceptable salt, ester, amide, hydrate, stereoisomer, or solvate thereof.

In another aspect, the present invention provides a compound of Formula I:

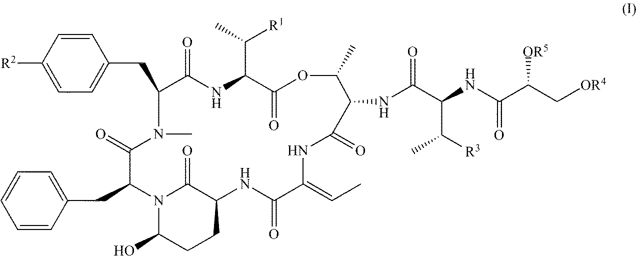

wherein:

R¹ is alkyl;

R² is H or hydroxyl;

R³ is alkyl or hydroxyl;

R⁴ is H or SO₃M⁺, wherein M⁺ is Na, K, or Li; and

R⁵ is H or alkyl;

with the proviso that when R¹ is alkyl, R² is H, R³ is alkyl, and R⁵ is alkyl,

then R⁴ is not H;

or a pharmaceutically acceptable salt, ester, amide, hydrate, stereoisomer, or solvate thereof.

In yet another embodiment, the compound is selected from any one of the compounds set forth below:

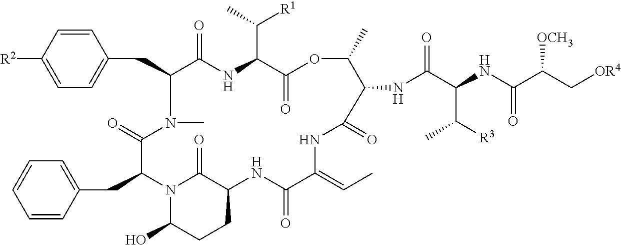

| | | R¹ | R² | R³ | R⁴ |
|---|---|---|---|---|---|
| Symplostatin 5 | (1) | Et | H | Me | SO₃Na |
| Symplostatin 6 | (2) | Me | H | Me | SO₃Na |
| Symplostatin 7 | (3) | Et | H | Et | SO₃Na |
| Symplostatin 8 | (4) | Et | OH | Me | SO₃Na |
| Symplostatin 9 | (5) | Me | OH | Me | SO₃Na |
| Symplostatin 10 | (6) | Et | OH | Et | SO₃Na |

In another embodiment, the compounds are those of formula (I), wherein R⁴ is SO₃Na.

In another embodiment, the compounds are those of formula (I), wherein R⁴ is SO₃M⁺.

In another embodiment, the compounds are those of formula (I), wherein R⁴ is SO₃Na; R¹ is independently alkyl; and R³ is independently alkyl.

### EXEMPLARY SYNTHESIS

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Compounds herein can be isolated from natural sources as described herein. Additional methods of synthesizing compounds of Formula I and their synthetic precursors, including those within routes not explicitly shown in schemes herein, are within the means of chemists of ordinary skill in the art. Methods for optimizing reaction conditions and, if necessary, minimizing competing by-products, are known in the art. In addition to the synthetic references cited herein, reaction schemes and protocols may be determined by the skilled artisan by use of commercially available structure-searchable database software, for instance, SciFinder® (CAS division of the American Chemical Society), STN® (CAS division of the American Chemical Society), CrossFire Beilstein® (Elsevier MDL), or internet search engines such as Google® or keyword databases such as the US Patent and Trademark Office text database.

The methods to make the compounds described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### COMPOSITIONS

The invention also provides compositions comprising an effective amount of a compound of Formula I (e.g., including any of the formulae herein), or a pharmaceutically acceptable salt, solvate, or hydrate of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as a compound of any of the formulae herein.

Preferably, the second therapeutic agent is an agent useful in the treatment or prevention of a disease or condition selected from chronic obstructive pulmonary disease (COPD), cystic fibrosis, cancer, lung tissue injury (emphysema), asthma, rheumatoid arthritis.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can vary dependent on the subject. Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective amount of a compound of the invention is expected to vary from about 0.005 μg/kg to about 200 mg/kg per day, or 0.001 milligram per kilogram of body weight per day (mg/kg/day) to about 100 mg/kg/day.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for A compound of any of the formulae herein.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

In another embodiment, the invention provides a method of modulating the activity of elastase in a cell, comprising contacting a cell with one or more compounds of Formula I herein.

According to another embodiment, the invention provides a method of treating a patient suffering from, or susceptible to, a disease that is beneficially treated by a compound of any of the formulae herein comprising the step of administering to said patient an effective amount of a compound or a composition of this invention. Such diseases (e.g., elastase modulated diseases or disorders) are well known in the art and are disclosed herein as well.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method). In other methods, the subject is prescreened or identified as in need of such treatment by assessment for a relevant marker or indicator of suitability for such treatment.

In another embodiment, any of the above methods of treatment comprises the further step of co-administering to said patient one or more second therapeutic agents. The choice of second therapeutic agent may be made from any second therapeutic agent known to be useful for co-administration with A compound of any of the formulae herein. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Kits

The present invention also provides kits for use to treat chronic obstructive pulmonary disease (COPD), cystic fibrosis, cancer, lung tissue injury (emphysema), asthma, rheumatoid arthritis, or to provide and/or enhance anti-aging properties of skin by preventing (e.g., UVA-induced) wrinkle formation. These kits comprise (a) a pharmaceutical composition comprising a compound of Formula I or a salt, hydrate, or solvate thereof, wherein said pharmaceutical composition is in a container; and (b) instructions describing a method of using the pharmaceutical composition to treat a disease or disorder in a subject (e.g., a disorder delineated herein).

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### EXAMPLES

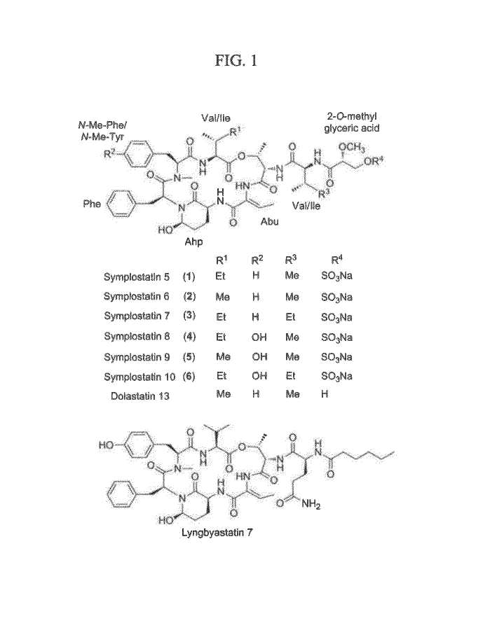

The following examples are provided by way of illustration and are not intended to limit the scope of the invention. The lyophilized red cyanobacterium collected from Cetti Bay, Guam was extracted with EtOAc-MeOH (1:1) to yield the nonpolar extract. This extract showed weak cytotoxic activity at a dosage of 10 μg/mL and no detectable largazole, symplostatin 1 or dolastatin 10, which we commonly find in Symploca collections. Liquid-liquid partitioning of the nonpolar extract yielded the hexanes-, n-BuOH— and H₂O-soluble fractions. The ¹H NMR spectrum of the n-BuOH fraction showed characteristic resonances for peptides and modified peptides. This fraction was further purified by silica column chromatography. ¹H NMR profiling of individual column fractions indicated that peptides and modified peptides eluted with 50% i-PrOH in CH₂Cl₂. Further purification of this peptide-enriched fraction by reversed-phase HPLC yielded six new compounds, termed symplostatins 5-10 (1-6) (FIG. 1).

The major compound, symplostatin 5 (1) (FIG. 1), showed a pseudomolecular ion of 1044.3981 [M+Na]⁺, suggesting a molecular formula of C₄₇H₆₄N₇O₁₅SNa. LRESIMS using negative ionization showed a loss of 46 amu (m/z 998.5 [M−Na]⁻) relative to the pseudomolecular [M+Na]⁺ ion observed in the positive ionization mode. This corresponds to loss of 2×Na ions and supported that 1 is present as a sodium salt. The ¹H NMR spectrum of symplostatin 5 (1) showed characteristic signals for peptides and modified peptides such as secondary amide protons (δ_{H} 8.18, 7.71, 7.40, 7.34), an N—CH₃ proton (δ_{H} 2.77), and α-protons (δ_{H} 3.80-5.10). Analysis of the COSY, TOCSY, HSQC and HMBC (Table 7) data acquired in DMSO-d₆, established the presence of Val, Thr, Ile, N-Me-Phe, and Phe. Among the three remaining spin systems, one is a distinctive methine quartet (δ_{H} 6.50) that showed a COSY correlation to a CH₃ doublet (δ_{H} 1.47) (Table 1). HMBC correlations of the latter to a carbonyl at δ_{C} 162.9 and a quaternary sp² C (δ_{C} 130.0), together with a TOCSY correlation to a broad NH singlet (δ_{H} 9.24), established this unit as 2-amino-2-butenoic acid (Abu), a modified (dehydrated) Ser residue. The observed low-field methine signal at δ_{C}/δ_{H} 73.4/5.03 together with a hydroxy proton resonating at δ_{H} 6.05 in 1 are distinctive for 3-amino-6-hydroxy-2-piperidone (Ahp) unit, a modified Glu residue. The presence of this cyclized amino acid residue was supported by COSY correlation of the α-proton (H-3) with diastereotopic methylene protons (H-4a and H-4b), together with correlations between H-4a/H-5a, H-4a/H-5b and H-5b/H-6 (Table 1). HMBC correlations to a carbonyl group (δ_{C} 168.7) were also observed H-3 (δ_{H} 3.75) and H-6 (δ_{H} 5.03). The last spin system consisted of a low-field methine (δ_{c}/δ_{H} 79.9/3.98), an oxygenated diastereotopic methylene (δ_{c}/δ_{H} 66.1/3.90, 3.73) and an —OCH₃ group (δ_{c}/δ_{H} 57.1/3.33). From COSY and HMBC analysis, this moiety corresponds to a modified glyceric acid, where the C-2 and C-3 positions are methoxylated and sulfated, respectively (Table 1). Sulfated glyceric acid and 2-O-methyl glyceric acid sulfate has been observed in other metabolites from freshwater and marine cyanobacteria.^{2, 3, 13, 1415} The ¹H and ¹³C NMR chemical shifts (DMSO-d₆) of this moiety are in close agreement with the reported values for the same residue in oscillapeptin F.¹³ The linear sequence of 2-O-methyl glyceric acid sulfate-Val-Thr-Abu-Ahp-N-Me-Phe-Phe-Ile was established using HMBC and NOESY correlations. The upfield shifted methyl group (δ_{H} 0.71) of Ile together with its NOESY correlation to H-6 of Phe (δ_{H} 7.39) suggested that this moiety is adjacent to the aromatic system. Furthermore, one of the methyl groups of Val (δ_{H} 0.88) showed NOESY correlation with the Abu methyl group (δ_{H} 1.47) indicating proximity in space between these functionalities. In order to fulfill the molecular formula requirements and to account for the low-field ¹H chemical shift of the vicinal methine of Thr (δ_{C}/δ_{H} 71.5, 5.52), additional anisotropic effect from a carbonyl group must be present and this indicated cyclization of symplostatin 5 (1) via the carbonyl group of Ile and the hydroxy group of Thr.

The HRESIMS data of symplostatin 6 (2) showed a peak at m/z 1030.3815, corresponding to the pseudomolecular ion [M+Na]⁺ and suggested a molecular formula of C₄₆H₆₂N₇O₁₅SNa. Comparison of the ¹H NMR spectrum of 1 and 2 revealed differences in the splitting pattern of signals in the methyl region (δ_{H} 0.75-0.90), and no methyl triplet arising from Ile was observed in the latter. Instead two pairs of methyl doublets were present, suggesting the presence of 2× Val units. This was corroborated by the HSQC spectrum of symplostatin 6 (2), where the characteristic high-field methyl carbon signal (δ_{C} 11.3) of Ile was absent. Hence, the Ile unit present in the core ring structure of 2 is replaced by Val, where the vicinal methine (δ_{C}/δ_{H} 30.7/2.00) showed COSY and HMBC correlations to two methyl groups (δ_{C}/δ_{H} 19.0/0.89, 17.1/0.76). Symplostatin 6 (2) is reminiscent of dolastatin 13, with the primary difference being the modification of the 2-O-methyl glyceric acid unit. Hence, symplostatin 6 (2) is the sulfated analog of dolastatin 13 (FIG. 1).

Symplostatin 7 (3) showed 14 amu and 28 amu mass difference with symplostatin 5 (1) and symplostatin 6 (2), respectively. Based on the observed pseudomolecular [M+Na]⁺ observed at m/z 1058.4104, symplostatin 7 (3) has a molecular formula of C₄₈H₆₆N₇O₁₅SNa. The ¹H NMR spectrum of 3 showed 2×CH₃ triplets (δ_{H} 0.92, 0.80) which correlated to two high-field carbons (δ_{C} 11.3, 10.7) based on the HSQC spectrum (Table 3). Hence, 3 has Ile moieties in both the pendant and macrocyclic ring structure. Comparison of the ¹H NMR spectrum of 1 and 3 corroborated this result, except for ¹H resonances belonging to the additional Ile unit, no significant differences were observed between the two spectra.

The HRESIMS spectrum of symplostatin 8 (4) showed a pseudomolecular ion [M+Na]⁺ peak at m/z 1060.3941, giving a 16 amu difference compared to symplostatin 5 (1), corresponding to an additional oxygen atom based on its molecular formula of C₄₆H₆₄N₇O₁₆SNa. The ¹H NMR spectra of these two compounds were highly similar, except for the splitting pattern and chemical shifts of aromatic protons (δ_{H} 6.77-7.40). Comparison of the HSQC spectra of these compounds showed an upfield shifted sp² C at δ_{C} 115.2 which correlates to a proton at δ_{H} 6.77 for 4. COSY correlation between δ_{H} 6.77 and δ_{H} 6.99 together with their doublet splitting pattern and ³J_{H,H} of 7.8 Hz indicated a 1,4-disubstituted phenyl ring in 4 (Table 1). The upfield shifted ¹H and ¹³C NMR resonances and the presence of a broad singlet at δ_{H} 9.34 for a hydroxy group in the ¹H NMR spectrum of 4 supported the presence of an N-Me-Tyr instead of an N-Me-Phe in the macrocycle. Hence, symplostatin 8 (4) is the N-Me-Tyr analog of 1 (FIG. 1), consistent with the molecular formula.

Comparison of the ¹H NMR spectra of symplostatin 9 (5) and symplostatin 10 (6) with 4 indicated that these compounds have similar aromatic regions (δ_{H} 6.77-7.40) and differ in the high-field region corresponding to signals from methyl groups (δ_{H} 0.75-0.90). This indicated that 5 and 6, like compound 4, both contain N-Me-Tyr. Hence, Val-Ile substitutions are expected between these congeners. Symplostatin 9 (5) (C₄₆H₆₂N₇O₁₆SNa) showed a 14 amu mass difference to 4 and lacked the characteristic ¹H and ¹³C NMR resonances corresponding to the methyl group of Ile. Hence, 5 possesses 2× Val units and is the N-Me-Tyr analog of 2 (FIG. 1). Symplostatin 10 (6) (C₄₈H₆₆N₇₆SNa) showed 14 amu and 28 amu mass difference to compounds 4 and 5, respectively. Inspection of the ¹H NMR and HSQC spectra indicated the presence of 2× Ile units in symplostatin 10 (6) based on characteristic high-field ¹H and ¹³C resonances for two CH₃ groups (δ_{C}/δ_{H} 11.3/0.91, 10.6/0.80). Hence, symplostatin 10 (6) is the N-Me-Tyr analog of symplostatin 7 (3).

Enantioselective HPLC-MS analysis of the acid hydrolysates of these six compounds established the configuration of the amino acids (Val, Phe, N-Me-Phe, N-Me-Tyr, Thr) as L-, by comparison to authentic standards. L-allo-Ile was detected for compounds having this unit in the macrocycle alone (1, 4). Compounds with Ile in both the macrocycle and pendant chain (3, 6) showed peaks corresponding to L-allo-Ile and L-Ile at ˜1:1 ratio. Comparison of the ¹H and ¹³C chemical shifts of the macrocyclic Ile unit of these four compounds showed no significant differences and suggested the same configuration for the macrocyclic Ile. Hence, the pendant side chain Ile moiety would then account for the peak corresponding to L-Ile. Oxidation of 1 using CrO₃ prior to acid hydrolysis converted the Ahp unit to Glu. Enantioselective analysis of the acid hydrolysate of the oxidation product showed a peak corresponding to L-Glu and hence, the Ahp unit would have the same configuration at C-3. The configuration at C-6 of Ahp is deduced to be R in comparison with the NMR chemical shifts with the related compounds symplostatin 2 and lyngbyastatins 4-10. The 2-O-methyl glyceric acid liberated from the acid hydrolysate of symplostatin 5 (1) is proposed to have an R-configuration, based on comparison with authentic standards of 2-O-methyl glyceric acid synthesized from D- and L-Ser by a modified diazotization procedure. The other analogs of symplostatin 5 (1) are proposed to have the same configuration of the Ahp and 2-O—CH₃ glyceric acid moieties based on similar ¹H and ¹³C chemical shifts.

Symplostatin 5 (1) was assessed for its ability to inhibit the proteolytic activity of porcine pancreatic elastase, chymotrypsin and trypsin by direct incubation of the purified enzymes together with a p-nitroanilidine conjugated peptide substrate. The absorbance of the in vitro enzyme assay over time is related to the liberated p-nitroanilidine and hence to the enzyme activity. Symplostatin 5 (1) gave IC₅₀ values of 68 nM and 217 nM for porcine pancreatic elastase and chymotrypsin, respectively. Trypsin was not inhibited by 1, up to the highest concentration (10 μM) tested.

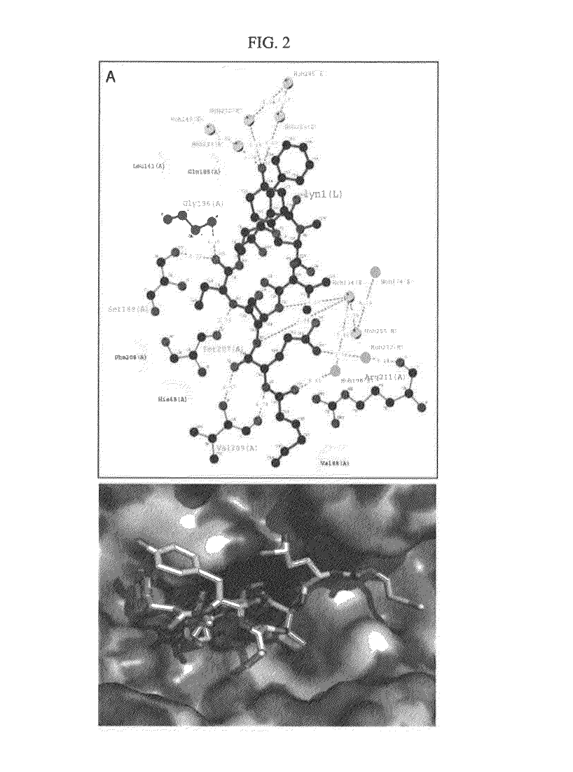

In order to understand the potent and selective inhibitory activity of the Abu-bearing lyngbyastatins and symplostatins against elastase, lyngbyastatin 7 and porcine pancreatic elastase were cocrystallized using the hanging drop technique. A cocrystal structure was obtained at a resolution of 1.5 Å, the best ever reported for elastase-inhibitor complex. The natural products scyptolin¹⁶ and FR901277¹⁷ were previously cocrystallized but at a lower resolution of 2.8 Å and 1.6 Å, respectively. Porcine pancreatic elastase, despite sharing only 40% amino acid sequence homology to human neutrophil elastase, is an accepted model system to understand key-enzyme inhibitor interactions.^{18, 19} Porcine pancreatic elastase and human neutrophil elastase are structurally comparable and share similar residues that compose the substrate binding site. The improved resolution of the elastase-lyngbyastatin 7 complex provided better insights into key molecular interactions with the enzyme. While several structurally related compounds to lyngbyastatin 7 have been reported to inhibit elastase, this is the first cocrystal structure obtained for this class of compounds. The cocrystal structure of porcine pancreatic elastase and lyngbyastatin 7 indicated that these compounds act as substrate mimics, with the Abu moiety and the N-terminal residues occupying subsites S1 to S4. Lyngbyastatin 7 showed an extensive array of hydrogen bonds and non-bonded interactions with elastase and several water molecules in the active site (FIG. 2). The ethylidene moiety of the Abu unit in subsite S1 contributes a CH/π interaction with Ser188 and together with hydrogen bonds formed with Ser207 and Gly188, can account for potent and selective elastase inhibitory activity of this family of compounds. The cocrystal structure did not show covalent bond formation between lyngbyastatin 7 and elastase, nor hydrolytic cleavage of the macrocycle. The intermolecular hydrogen bonds formed between NH (Phe) and carbonyl (Val) and NH (Val) and OH (Ahp) creates a rigid cyclic depsipeptide core in lyngbyastatins and related compounds, hindering proteolytic cleavage by elastase. The key molecular interactions between the ethylidene moiety of lyngbyastatin 7 and elastase corroborate the observed anti-proteolytic activity of Abu-containing symplostatins, dolastatins, and lyngbyastatins and indicative of similar binding interactions with the enzyme. It is surprising that the pendant side chain, which is highly divergent among these compounds, participates in extensive enzyme-inhibitor interaction and highlights Nature's own chemical optimization. Lyngbyastatins 8-10 are less potent elastase inhibitors, with reported IC₅₀s of 120-210 nM,⁴ suggesting that the presence of mainly hydrophobic residues in the pendant side chain is unfavorable. This is supported by our cocrystal structure, wherein polar functional groups such as the side chain NHs of Lys can participate in indirect hydrogen bonding interactions with Arg211, via a water molecule, which would be unfavorable with nonpolar functionalities. The structural information obtained here can be utilized as a guide for improvements in the design of inhibitors based on the dolastatin 13 scaffold, with the possibility of modulating the activity through modification of the pendant side chain.

The anti-proteolytic activity of symplostatin 5 (1) and lyngbyastatin 7 on elastase are comparable (IC₅₀ values of 68 nM and 47 nM, respectively). Hence, 1 is expected to have similar binding modes with elastase as with lyngbyastatin 7. Symplostatin 5 (1) was used in subsequent bioassays to probe the cellular effects of this class of compounds.

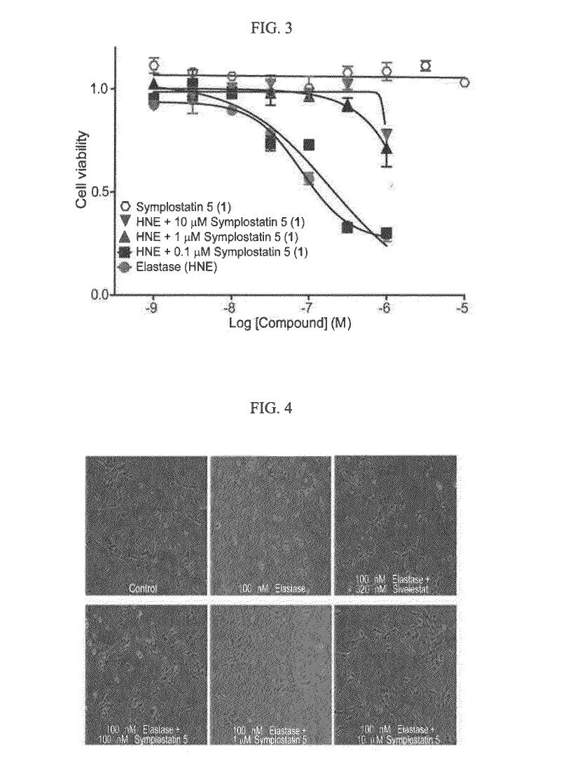

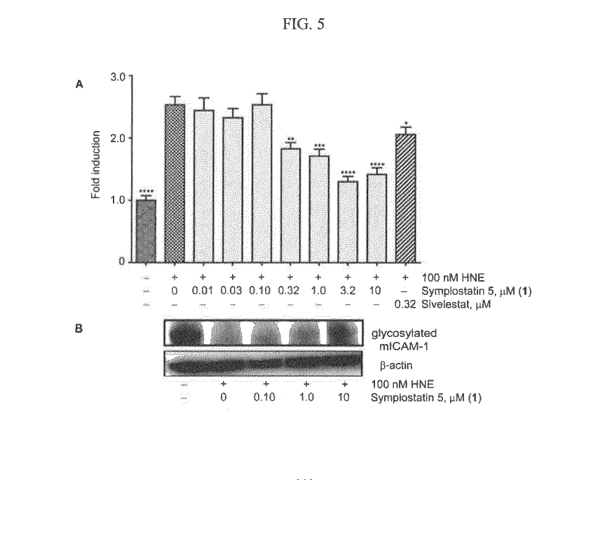

Elastase is the primary enzyme involved in the degradation of elastin and released by neutrophils in response to tissue injury. Increased levels of elastase have been reported in chronic obstructive lung disease, asthma, artherosclerosis and autoimmune diseases, through either increased levels of the enzyme or downregulated expression of its endogenous inhibitors.^{11, 12} Unregulated elastase activity is linked to direct cytotoxicity to the cells, cytokine processing, activation of receptors and cleavage of adhesion molecules.²⁰ As expected, symplostatin 5 (1) showed comparable anti-proteolytic activity on both human neutrophil elastase and porcine pancreatic elastase. To determine the cellular effects of elastase inhibition mediated by the lyngbyastatins and symplostatins and simulate disease etiology, bronchial epithelial cells were challenged with human neutrophil elastase and co-treated with either DMSO or varying concentrations of symplostatin 5 (1). Elastase exhibited anti-proliferative activity within 24 h of treatment of bronchial epithelial cells, with an IC₅₀ of 82 nM (FIG. 3). Elastase was previously demonstrated to induce apoptosis via a PAR-1-, NF-κB- and p53-dependent pathway in lung epithelial cells.²¹ The anti-proliferative effect of elastase was alleviated by symplostatin 5 (1) treatment at dosages of 1 and 10 μM. Symplostatin 5 (1) did not show any antiproliferative effects on bronchial epithelial cells when administered alone at all the dosages tested (FIG. 3).

An apparent change in cellular morphology of bronchial epithelial cells was observed within 2 h of elastase treatment, with cells becoming rounded, in contrast to the elongated appearance of control cells (FIG. 4). Symplostatin 5 (1) restored the elongated cellular morphology of elastase-treated cells at dosages of >100 nM. These observations indicated intercellular adhesion molecules as a possible direct target of exogenous elastase in bronchial epithelial cells. The regulation of intercellular adhesion molecule-1 (ICAM-1) expression, while being mainly transcriptional, is also suggested to involve posttranslational events.^{22, 23} ICAM-1, which exists as a membrane-bound protein, can be cleaved to generate soluble ICAM-1. Several proteases have been implicated in the posttranslational modification of membrane-bound ICAM-1 (mICAM-1) to soluble ICAM-1 (sICAM-1), depending on the cell type. In endothelial, fibroblast and astrocyte cultures, sICAM-1 generation is mediated by a matrix metalloprotease or ADAM 17²⁴.

In contrast, sICAM-1 production in alveolar epithelial cells was reportedly inhibited by the serine protease inhibitor aprotinin, although the specific enzyme responsible was not identified.²⁵ In a separate experiment, ICAM-1 was shown to undergo proteolytic cleavage in the presence of elastase and also upregulate ICAM-1 transcript levels.^{10, 26}

To demonstrate the effect of elastase and symplostatin 5 (1) treatments on ICAM-1 expression in bronchial epithelial cells, culture supernatants and cell lysates were collected and monitored by AlphaLisa® and immunoblotting, respectively. Bronchial epithelial cells that were challenged with elastase showed an increase in sICAM-1 levels, relative to control supernatants. Cotreatment with symplostatin 5 (1) showed a dose-dependent decrease in sICAM-1 production, with significant downregulation of sICAM-1 levels at concentrations of >100 nM, the same effective concentration that prevented cytotoxicity of elastase. The elastase inhibitor sivelestat (Elaspol®) also decreased sICAM-1 at a dosage of 320 nM. To correlate that the liberated sICAM-1 is generated from the proteolysis of mICAM-1 by elastase, whole cell lysates from elastase-stimulated cells treated with DMSO or symplostatin 5 (1) were probed with an ICAM-1 specific antibody. A significant difference in expression of fully glycosylated mICAM-1 (˜100 kDa) was observed between the control and elastase treatment, with maximum expression in the former. Conversely, an increase in mICAM-1 levels in elastase-stimulated cells was attained with cotreatment of 10 μM of symplostatin 5 (1). This inverse relationship is consistent with measurements of sICAM-1 levels in the culture supernatants and provided internal validation of the direct effects of elastase with the proteolytic cleavage of ICAM-1.

The observed morphological changes, cell death and increased soluble ICAM-1 levels has been reported in allergic inflammation and asthma.^{24, 27} The level of sICAM-1 is higher in asthmatics and related to the activation of cationic protein-secreting eosinophils that can trigger cell damage and toxicity.²⁴ While the Abu-containing lyngbyastatins, dolastatins and symplostatins can alleviate the direct cytotoxic effects of elastase on bronchial epithelial cells, these cyanobacteria-derived metabolites can also effectively modulate the downstream pathways altered by elastase and may have potential application in allergic inflammation and asthma. Hitherto, this is the first report of the cellular effects and molecular interactions of symplostatins and lyngbyastatins with elastase. The observed cellular effects of symplostatin 5 (1) are comparable to that of sivelestat, which is in clinical trial for acute lung injury. Furthermore, both compounds appear to be equipotent in the systems used in this study.

### EXPERIMENTAL SECTION

### General Experimental Procedures

Optical rotations were measured on a Perkin-Elmer 341 polarimeter. UV spectra were recorded on SpectraMax MS (Molecular Devices). ¹H and 2D NMR spectra were recorded in CDCl₃ on a Bruker Avance II 600 MHz spectrometer equipped with a 5-mm TXI cryogenic probe using residual solvent signals [(CDCl₃: δ_{H} 7.26; δ_{C} 77.0), (DMSO-d₆: δ_{H} 2.50; δ_{C} 39.5)] as internal standards. HSQC and HMBC experiments were optimized for ¹J_{CH}=145 and ⁿJ_{CH}=7 Hz, respectively. TOCSY experiments were done using a mixing time of 100 ms. ¹³C NMR spectra were recorded on Varian 400 MHz or Bruker 500 MHz NMR spectrometers. HRESIMS data was obtained using an Agilent LC-TOF mass spectrometer equipped with an APCI/ESI multimode ion source detector. LRESIMS measurements, MRM analysis and MS/MS fragmentation were done on an ABI 3200Q TRAP.

### Biological Material

The red Symploca sp. cyanobacterium collection was collected by hand from Cetti Bay, Guam. Samples were kept frozen at −20° C. after collection. A voucher specimen, which is preserved in 100% EtOH or formaldehyde, is deposited in the University of Guam Herbarium and at the Smithsonian Marine Station, Fort Pierce, Fla. Frozen cyanobacterium samples were lyophilized prior to extraction.

### Extraction and Isolation

The freeze-dried cyanobacterium was extracted with EtOAc-MeOH (1:1) to yield the nonpolar extract. This was partitioned between hexanes and 20% aqueous MeOH, the latter concentrated under reduced pressure and further partitioned between n-BuOH and H₂O. The n-BuOH fraction was concentrated to dryness and chromatographed on Si gel eluting first with CH₂Cl₂, followed by increasing concentrations of i-PrOH, while after 100% i-PrOH, increasing gradients of MeOH were used.

The fraction collected from 50% i-PrOH elution (Si column) was purified by C18 column eluting with 25%, 50%, 75% and 100% MeOH in H₂O. The fraction from 50% MeOH was further purified using semipreparative reversed-phase HPLC (Phenomenex Synergi-Hydro RP, 4 μm; flow rate, 2.0 mL/min) using a linear gradient of MeCN—H₂O (25%-100% MeOH in 30 min and then 100% MeCN for 10 min) to yield compounds 5 (t_{R} 17.8 min, 1.0 mg), 4 (t_{R} 19.0 min, 1.0 mg) and 6 (t_{R} 36.6 min, 0.3 mg). The 75% MeOH fraction was purified using the same HPLC conditions to yield compounds 2 (t_{R} 23.8 min, 3.5 mg), 1 (t_{R} 24.0 min, 7.0 mg) and 3 (t_{R} 25.3 min, 1.0 mg).

Symplostatin 5 (1):

colorless, amorphous solid; [α]²⁰_{D} −3.6 (c 0.14, MeOH); UV (MeOH); λₘₐₓ (log ε) 210 (4.49); ¹H NMR, ³C NMR, COSY, and HMBC data, see Table 1; HRESIMS m/z 1044.3981 [M+Na]⁺ (calcd for C₄₇H₆₄N₇O₁₅SNa, 1044.3971).

Symplostatin 6 (2):

colorless, amorphous solid; [α]²⁰_{D} −5.2 (c 0.26, MeOH); UV (MeOH); λₘₐₓ (log ε) 204 (4.49); ¹H NMR and ¹³C NMR data, see Table 2; HRESIMS m/z 1030.3815 [M+Na]⁺ (calcd for C₄₆H₆₂N₇O₁₅SNa, 1030.3815).

Symplostatin 7 (3):

colorless, amorphous solid; [α]²⁰_{D} −14 (c 0.15, MeOH); UV (MeOH); λₘₐₓ (log ε) 202 (4.48); ¹H NMR and ¹³C NMR data, see Table 3; HRESIMS m/z 1058.4104 [M+Na]⁺ (calcd for C₄₆H₆₄N₇O₁₆SNa, 1058.4128).

Symplostatin 8 (4):

colorless, amorphous solid; [α]²⁰_{D} −6.7 (c 0.09, MeOH); UV (MeOH); λₘₐₓ (log ε) 210 (4.13); ¹H NMR and ¹³C NMR data, see Table 1; HRESIMS m/z 1060.3941 [M+Na]⁺ (calcd for C₄₆H₆₄N₇O₁₆SNa, 1060.3920).

Symplostatin 9 (5):

colorless, amorphous solid; [α]²⁰_{D} −3.5 (c 0.10, MeOH); UV (MeOH); λₘₐₓ (log ε) 204 (3.94); ¹H NMR and ¹³C NMR data, see Table 2; HRESIMS m/z 1046.3747 [M+Na]⁺ (calcd for C₄₆H₆₄N₇O₆SNa, 1046.3764).

Symplostatin 10 (6):

colorless, amorphous solid; [α]²⁰_{D} −3.3 (c 0.03, MeOH); UV (MeOH); λₘₐₓ (log ε) 200 (5.24); ¹H NMR and ¹³C NMR data, see Table 3; HRESIMS m/z 1074.4060 [M+Na]⁺ (calcd for C₄₈H₆₆N₇O₆SNa, 1074.4077).

Enantioselective Analysis.

Portions of 1-6 (100 μg) were acid-hydrolyzed (200 μL 6 N HCl, 110° C., 20 h), the product mixtures dried, reconstituted in 100 μL H₂O and analyzed by enantioselective HPLC-MS. The absolute configurations of the amino acids (Ile, Val, N-Me-Tyr, N-Me-Phe, Phe, Thr) were determined by enantioselective HPLC-MS [column, Chirobiotic TAG (250× 4.6 mm), Supelco; solvent, MeOH-10 mM NH₄OAc (40:60, pH 5.30); flow rate, 0.5 mL/min; detection by ESIMS in positive ion mode (MRM scan)]. The retention times (t_{R}, min; MRM ion pair) of the authentic amino acids were as follows: L-Val (7.8; 118→72), D-Val (13.7); L-Ile (8.4; 132→86), L-allo-Ile (8.6), D-allo-Ile (17.6), D-Ile (20.2); N-Me-L-Phe (22.7; 180→134), N-Me-D-Phe (40.4), L-Phe(12.1; 166→103), D-Phe (17.5), N-Me-L-Tyr (18.8; 196→77), N-Me-D-Tyr(35.4), L-Thr (6.8; 120→74), L-allo-Thr (7.2), D-Thr (8.0), D-allo-Thr (10.2).

The modified glyceric acid residues 2-O—CH₃ glyceric acid was prepared using L-Ser (50 mg), isoamyl nitrite (70.5 μL), glacial CH₃COOH (17.2 μL), MgSO₄ (60 mg) and anhydrous MeOH (1 mL). The mixture was heated at 110° C. for 4 h in a tightly sealed vial. The reaction was cooled down to room temperature and filtered. The filtrate was evaporated to dryness under N₂ to yield 2S—O-methyl glyceric acid. The same procedure was employed to prepare 2R—O-methyl glyceric acid from D-Ser. LRESIMS and ¹H NMR spectrum for 2R—O-methyl glyceric acid and 2S—O-methyl glyceric acid were in agreement with reported literature values.

The absolute configuration of Glu and 2-O-methyl glyceric acid was determined using the same HPLC conditions with detection in the negative ion mode (MRM) instead. The retention times of the authentic standards (t_{R}, min; MRM pair): L-Glu (5.1; 146→102), D-Glu (6.1), 2S—O-methyl glyceric acid (6.1; 119→89), 2R—O-methyl glyceric acid (6.6). Compound 1 was oxidized using ClO₃ and hydrolyzed using 6 N HCl (110° C., 20 h) to convert Ahp to Glu. The oxidized acid hydrolysate of 1 showed a peak at 5.1 min corresponding to L-Glu. The acid hydolysate peak of 1 showed a peak for 2R—O-methyl glyceric acid (t_{R} 6.6 min).

The acid hydrolysates of 1-6 showed retention times at 6.8 and 12.1 min corresponding to L-Thr, L-Phe. L-Val (t_{R} 7.8 min) was detected in the acid hydrolysates of 1, 2, 4, and 5. A peak corresponding to L-Ile (t_{R} 8.4 min) was observed in the acid hydrolysates of 1, 3, 4, and 6. In addition 4 and 6 had an additional peak at 8.6 min corresponding to L-allo-Ile. N-Me-L-Phe (t_{R} 22.7 min) was detected in the acid hydrolysate of 1-3, while N-Me-L-Tyr (t_{R} 18.8 min) was present in the acid hydrolysate of 4-6.

### Biological Activities of Symplostatin 5-10 (1-6)

### Example 1

### Cell Viability Assay

Bronchial epithelial cells (BEAS-2B, ATCC) were grown in bronchial epithelial basal media (BEBM, Lonza) supplemented with bronchial epithelial growth factors (Lonza) under a humidified environment with 5% CO₂ at 37° C. BEAS-2B (5,000) cells were seeded in collagen-coated 96-well plates and treated with varying concentrations of elastase after 24 h of seeding. These were either co-treated with varying doses of symplostatin 5 (1) or solvent control (DMSO). The cells were incubated for an additional 24 h before the addition of the MTT reagent. Cell viability was measured according to the manufacturer's instructions (Promega). IC₅₀ calculations were done by GraphPad Prism 5.03 based on duplicate experiments.

### Example 2

### Serine Protease Inhibition Assay

Porcine pancreatic elastase (Elastase-high purity; EPC, EC134) was dissolved in Tris-HCl (pH 8.0) to give a concentration of 75 μg/mL. Test compounds (1 μL, DMSO), 5 μL elastase solution (75 μg/mL) and 79 μL Tris-HCl (pH 8.0) were preincubated at ambient temperature for 15 min in a 96-well microtiter plate. At the end of the incubation, 15 μL substrate solution was added (2 mM N-succinyl-Ala-Ala-Alap-nitroanilide in Tris-HCl, pH 8.0) to each well, and the reaction was monitored by taking the absorbance at 405 nm every 30 s. The inhibitory activity of symplostatin 5 (1) against human neutrophil elastase was also determined using the same procedure with minor modifications, using 100 μg/mL human neutrophil elastase and 2 mM N-succinyl-Ala-Ala-Pro-Valp-nitroanilide, both prepared in 0.1 M Tris-NaCl buffer (pH 7.5). Enzyme activity was determined by calculating the initial slope of each progress curve, expressed as a percentage of the slope of the uninhibited reaction. Chymotrypsin and trypsin activity with symplostatin 5 (1) treatment were assessed using a similar procedure as for elastase. N-Suc-Gly-Gly-Phe-p-nitroanilide and Na-Benzoyl-DL-arginine 4-nitroanilide was used as substrates for chymotrypsin and trypsin, respectively.

### Example 3

### Measurement of sICAM-1 Levels

BEAS-2B cells (60,000) were seeded in collagen-coated 24-well plates. After overnight incubation, the media was replaced with supplement-free media and the cells were further incubated for 24 h. At the end of the incubation period, cells were replenished with new supplement-free media prior to treatment. Stock human neutrophil elastase solution was prepared in sodium acetate and test aliquots were further diluted in supplement-free medium. Cells were treated with elastase together with DMSO or varying concentrations of symplostatin 5 (1) dissolved in DMSO. Control cells were treated with DMSO (1%) and sodium acetate in supplement-free medium (4%). The cells were incubated for 6 h. Culture supernatants were collected and sICAM-1 levels were determined using ICAM-1 AlphaLisa® Kit (Perkin Elmer) according to the manufacturer's instruction.

### Example 4

### Immunoblot Analysis of mICAM-1 Levels

BEAS-2B cells (150,000) were grown in collagen-coated 6-cm tissue culture dishes. The supplemented media was replaced with BEBM after overnight incubation and further left to acclimatize for in supplement-free media. Cells were replenished with fresh BEBM and treated with elastase together with DMSO or varying concentrations of symplostatin 5 (1). Cells were harvested and lysed with Phosphosafe (Novagen, Madison, Wis.), 6 h posttreatment. The protein concentration of whole cell lysates was measured with the BCA Protein Assay kit (Pierce Chemical, Rockford, Ill.). Equal amounts of protein were separated by SDS-polyacrylamide gel electrophoresis (4-12%), transferred to polyvinylidene difluoride membranes, probed with antiICAM-1 antibody (Abcam) and detected with the SuperSignal West Femto Maximum Sensitivity Substrate (Pierce Chemical). The immunoblots were stripped by heating in a water bath (90° C.) and reprobed with antiβ-actin antibody to confirm equal protein loading.

### Example 5

### Co-Crystallization of Lyagbyastatin 7 with Porcine Pancreatic Elastase

A 10 μL aliquot of high purity porcine pancreatic elastase:lyngbyastatin 7 solution (3:1) was incubated in a hanging drop setup equilibrated against a 0.42 M sodium sulfate solution. The crystals were allowed to grow and data were collected to 1.5 Å resolution.

### REFERENCES

(1) Tan, L. T. J. Appl. Phycol. 2010, 22, 659-676.
(2) Matthew, S.; Ross, C.; Rocca, J. R.; Paul, V. J.; Luesch, H. J. Nat. Prod. 2007. 70, 124-127.
(3) Taori, K.; Matthew, S.; Rocca, J. R.; Paul, V. J.; Luesch, H. J. Nat Prod. 2007. 70, 1593-1600.
(4) Kwan, J. C.; Taori, K.; Paul, V. J.; Luesch, H. Mar. Drugs 2009, 7, 528-538.
(5) Taori, K.; Paul, V. J.; Luesch, H. J. Nat. Prod. 2008, 71, 1625-1629.
(6) Nogle, L. M.; Williamson, R. T.; Gerwick, W. H. J. Nat. Prod. 2001, 64, 716-719.
(7) Pettit, G. R.; Kamano, Y.; Herald, C. L.; Dufresne, C.; Cerny, R. L.; Herald, D. L.; Schmidt, J. M.; Kizu, H. J. Am. Chem. Soc. 1989, 111, 5015-5017.
(8) Harrigan, G. G.; Luesch, H.; Yoshida, W. Y.; Moore, R. E.; Nagle, D. G.; Paul, V. J. J. Nat. Prod. 1999, 62, 655-658.
(9) Kuwahara, I.; Lillehoj, E. P.; Lu, W.; Singh, I. S.; Isohama, Y.; Miyata, T.; Kim, K. C. Am J Physiol Lung Cell Mol Physiol 2006. 291, L407-IA 16.
(10) Ishihara, K.; Yamaguchi, Y.; Uchino, S.; Furuhashi, T.; Yamada, S.; Kihara, S.; Mori, K.; Ogawa, M. Dig Dis Sci 2006, 51, 2102-2112.
(11) Chua, F.; Laurent, G. J. Proc Am Thorac Soc 2006, 3, 424-427.
(12) Satoa, T.; Takahashi, S.; Mizumoto, T.; Harao, M.; Akizukid, M.; Takasugi, M.; Fukutomi, T.; Yamashita, J. Surg Oncol 2006, 15, 217-222.
(13) Itou, Y.; Ishida, K.; Shin, H. J.; Murakami, M. Tetrahedron 1999, 55, 6871-6882.
(14) Okumura, H. S.; Philmus, B.; Portmann, C.; Hemscheidt, T. K. J. Nat. Prod. 2009. 72, 172-176.
(15) Matthew, S.; Schupp, P. J.; Luesch, H. J. Nat. Prod. 2008, 71, 1113-1116.
(16) Matern, U.; Schelberger, C.; Jelakovic, S.; Weckesser, J.; Schulz, G. E. Chem. Biol. 2003, 10, 997-1001.
(17) Nakanishi, I.; Kinoshita, T.; Sato, A.; Tada, T. Bioploymers 2000, 53, 434-445.
(18) Sinha, S.; Watorek, W.; Karr, S.; Giles, J.; Bode, W.; Travis, J. Proc Natl Acad Sci USA 1987, 84, 2228-2232.
(19) Bode, W.; Meyer, E.; Powers, J. C. Biochemistry 1989, 28, 1951-1963.
(20) Pham, C. T. N. Nature Revs Immunol 2006. 6, 541-550.
(21) Suzuki, T.; Yamashita, C.; Zemans, R. L.; Briones, N.; van Linden, A.; Downey, G. P. Am J Respir Cell Mol Biol 2009. 41, 742-745.
(22) van de Stolpe, A.; van der Saag, P. T. J Mol Med 1996, 74, 13-33.
(23) Witkowska, A. M.; Borawska, M. H. Eur Cytokine Netw 2004. 15, 91-98.
(24) Stanciu, L. A.; Djukanovic, R. Eur Respir J 1998. 11, 949-957.
(25) Mendez, M. P.; Morris, S. B.; Wilcoxen, S.; Du, M.; Monroy, Y. K.; Remmer, H.; Murphy, H.; Christensen, P. J.; Paine III, R. Am J Physiol Lung Cell Mol Physiol 2007, 294, L807-L814.
(26) Champagne, B.; Tremblay, P.; Cantin, A.; St Pierre, Y. J Immunol 1998, 161, 6398-63405.
(27) Trautman, A.; Kruger, K.; Akdis, M.; Muller-Wenning, D.; Akkaya, A.; Brocker, E.; Blaser, K.; Akdis, C. A. Int Arch Allergy Immunol 2005, 138, 142-150.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

TABLE 1
NMR data of symplostatin 5 (1) and symplostatin 8 (4) in DMSO-d₆.

| | C/H | 1 | | | | 4 | |
|---|---|---|---|---|---|---|---|
| unit | no | δ_{C}^{a} | δ_{H} (J in Hz)^{b} | COSY^{b} | HMBC^{b} | δ_{C}^{a} | δ_{H} (J in Hz)^{b} |
| Ile | 1 | 170.0, C | | | | [symbol] | |
| | 2 | 54.0, CH | 4.89, br | NH | 1 | 54.0, CH | 4.87, d (11.0) |
| | 3 | 37.5, CH | 1.86, m | H₃-6 | | 37.0, CH | 1.86, m |
| | 4a | 25.8, CH₂ | 1.30, m | H-4b, H₃-5 | 2, 3 | 26.0, CH₂ | 1.29, m |
| | 4b | | 1.11, m | H-4a, H₃-5 | 2, 3 | | 1.12, m |
| | 5 | 11.2, CH₃ | 0.92, t (7.2) | H-4a, H-4b | 3 | 11.4, CH₃ | 0.91, t (7.3) |
| | 6 | 14.1, CH₃ | 0.71, d (7.0) | H-3 | 2, 3 | 14.3, CH₃ | 0.70, d (6.8) |
| | NH | | 7.40, br | H-2 | | | 7.40, br |
| N—Me-Phe^{c}/ | 1 | 172.7, C | | | | [symbol] | |
| N—Me-Tyr^{d} | 2 | 60.2, CH | 5.00, br | H-3a, H-3b | 1 | 60.7, CH | 4.90, d (10.6) |
| | 3a | 33.4, CH₂ | 3.23, brd (13.5) | H-2, H-3b | 4, 5/9 | 32.7, CH₂ | 3.11, d (−14.2) |
| | 3b | | 2.84, m | H-2, H-3a | 5/9 | | 2.70, dd (−14.2, 10.6) |
| | 4 | 137.9, C | | | | [symbol] | |
| | 5/9 | 129.4, CH | 7.23, d (7.5) | H-6 | | 130.3, CH | 6.99, d (7.8) |
| | 6/8 | 128.4, CH | 7.39, m | H-5, H-7 | 4 | 115.2, CH | 6.77, d (7.8) |
| | 7 | 126.5, CH | 7.30, m | H-6 | | [symbol] | |
| | OH | | | | | | 8.13, br s |
| | N—Me | 30.1, CH₃ | 2.77, s | | 2, 1 (Phe) | 30.3, CH₃ | 2.75, s |
| Phe | 1 | 170.3, C | | | | | |
| | 2 | 49.6, CH | 4.70, dd (11.4, 4.7) | H-3a, H-3b | 1, 2 (Ahp) | 50.0, CH | 4.73, m |
| | 3a | 34.8, CH₂ | 2.84, dd (−14.7, 11.4) | H-2, H-3b | 4 | 34.6, CH₂ | 2.87, dd (−14.2, 11.3) |
| | 3b | | 1.68, m | H-2, H-3a | 4 | | 1.81, m |
| | 4 | 136.5, C | | | | [symbol] | |
| | 5/9 | 129.2, CH | 6.77, d (7.5) | H-6 | 7 | 129.3, CH | 6.84, d (7.3) |
| | 6 | 127.6, CH | 7.18, m | H-5, H-7 | 4 | 127.7, CH | 7.19, m |
| | 7 | 126.1, CH | 7.15, m | H-6 | | 126.2, CH | 7.15, m |
| Ahp | 2 | 168.7, C | | | | [symbol] | |
| | 3 | 47.8, CH | 3.75, m | H-4a, H-4b, NH | 2 | 48.0, CH | 3.79, m |
| | 4a | 21.7, CH₃ | 2.38, m | H-3, H-4b, H-5a | | 21.9, CH₂ | 2.41, m |
| | 4b | | 1.56, m | H-3, H-4a | | | 1.58, m |
| | 5a | 29.0, CH₂ | 1.68, m | H-4a, H-5b, H-6 | | 29.2, CH₂ | 1.71, m |
| | 5b | | 1.50, m | H-5a, H-6 | | | 1.56, m |
| | 6 | 73.4, CH | 5.03, br s | H-5a, H-5b, OH | 2 | 73.5, CH | 5.07, m |
| | OH | | 6.05, s | H-6 | | | 6.07, br s |
| | NH | | 7.34, br | H-3 | | | 7.33, br |
| Abu | 1 | 162.9, C | | | | [symbol] | |
| | 2 | 130.0, C | | | | [symbol] | |
| | 3 | 131.7, CH | 6.50, q (7.2) | H₃-4 | 1, 4 | 131.5, CH | 6.49, q (7.2) |
| | 4 | 12.8, CH₃ | 1.47, d (7.2) | H-3 | 1, 2 | 13.0, CH₃ | 1.47, q (7.2) |
| | NH | | 9.24, br s | | | | 9.21, brs |
| Thr | 1 | [symbol] | | | | [symbol] | |
| | 2 | 55.1, CH | 4.67, br | NH | | 55.2, CH | 4.67, m |
| | 3 | 71.5, CH | 5.52, br s | H₃-4 | | [symbol] | 5.53, brs |
| | 4 | 17.5, CH₃ | 1.22, d (6.5) | H-3 | 2 | 17.7, CH₃ | 1.22, d (6.2) |
| | NH | | 8.18, br s | H-2 | | | 7.70, br s |
| Val | 1 | 172.2, C | | | | [symbol] | |
| | 2 | 56.4, CH | 4.47, t (7.2) | NH | 1 | 56.7, CH | 4.47, t (7.3) |
| | 3 | 30.7, CH | 2.09, m | H₃-4, H₃-5 | | 30.6, CH | 2.09, m |
| | 4 | 18.9, CH₃ | 0.88, d (6.7) | H-3 | 1 | 19.1, CH₃ | 0.88, d (7.0) |
| | 5 | 17.5, CH₃ | 0.83, d (6.7) | H-3 | 1 | 17.5, CH₃ | 0.83, d (7.0) |
| | NH | | 7.71, br s | H-2 | | | 7.72, br s |
| 2-O—CH₃ | 1 | 168.9, C | | | | [symbol] | |
| Glyceric | 2 | 79.9, CH | 3.98, dd (7.4, 3.4) | H-3a, H-3b | | 80.2, CH | 3.97, dd (7.3, 3.4) |
| Acid | 3a | 66.1, CH | 3.90, dd (−10.8, 3.4) | H-2, H-3b | | 66.2, CH | 3.89, dd (−10.7, 3.3) |
| | 3b | | 3.73, m | H-2, H-3a | | | 3.72, m |
| | OCH₃ | 57.1, CH₃ | 3.33^{g} | | 2 | 57.3, CH₃ | 3.32^{g} |

^{a}Deduced from HSQC and HMBC, 600 MHz.
^{b}600 MHz.
^{c}Refers to symplostatin 5 (1).
^{d}Refers to symplostatin 8 (4).
^{e}Not determined, predicted to have comparable chemical shifts based on highly homologous structures.
^{f}No correlation observed from HMBC.
^{g}Overlapping with residual water. [symbol]
indicates data missing or illegible when filed

TABLE 2
NMR data of symplostatin 6 (2) and symplostatin 9 (5) in DMSO-d [symbol] .

| | C/H | 2 | | 5 | |
|---|---|---|---|---|---|
| unit | no | δ_{C} [symbol] | δ_{H} (J in Hz)^{b} | δ_{C} [symbol] | δ_{H} (J in Hz)^{b} |
| Val | 1 | 170.2, C | | [symbol] | |
| | 2 | 56.0, CH | 4.68, m | 55.7, CH | 4.70, m |
| | 3 | 30.7, CH | 2.00, m | 30.3, CH | 2.08, m |
| | 4 | 19.0, CH₃ | 0.89, d (6.8) | 18.8, CH₃ | 0.88, d (6.8) |
| | 5 | 17.1, CH₃ | 0.76, d (6.8) | 17.0, CH₃ | 0.75, d (6.8) |
| | NH | | 7.51, d (8.8) | | 7.48, d (8.1) |
| N—Me-Phe^{b}/ | 1 | 169.3, C | | [symbol] | |
| N—Me-Tyr^{c} | 2 | 60.3, CH | 5.01, d (−11.3) | 60.5, CH | 4.89, d (10.9) |
| | 3a | 33.4, CH₃ | 3.23, m | 32.3, CH₂ | 3.10, d (−14.2) |
| | 3b | | 2.85, m | | 2.71, m |
| | 4 | 137.9, C | | [symbol] | |
| | 5/9 | 129.4, CH | 7.23, d (7.9) | 130.1, CH | 6.99, d (8.4) |
| | 6/8 | 128.4, CH | 7.39, m | 114.8, CH | 6.77, d (8.4) |
| | 7 | 126.5, CH | 7.30, m | | |
| | N—Me | 30.2, CH₃ | 2.79, s | 29.9, CH₃ | 2.76, s |
| | OH | | | | |
| Phe | 1 | 170.3, C | | [symbol] | |
| | 2 | 49.7, CH | 4.71, dd (−11.8, 4.4) | 49.8, CH | 4.73, dd (11.4, 3.8) |
| | 3a | 34.9, CH₂ | 2.85, m | 34.9, CH₂ | 2.87, dd (−14.4, 11.4) |
| | 3b | | 1.69, m | | 1.81, dd (−14.4, 3.8) |
| | 4 | 136.5, C | | [symbol] | |
| | 5/9 | 129.1, CH | 6.77, d (7.5) | 129.1, CH | 6.84, d (7.6) |
| | 6 | 127.6, CH | 7.18, m | 127.4, CH | 7.19, m |
| | 7 | 126.0, CH | 7.14, m | 126.0, CH | 7.14, m |
| Ahp | 2 | 168.7, C | | [symbol] | |
| | 3 | 47.9, CH | 3.76, m | 47.7, CH | 3.78, m |
| | 4a | 21.6, CH₂ | 2.42, m | 21.5, CH₂ | 2.42, m |
| | 4b | | 1.56, m | | 1.57, m |
| | 5a | 29.1, CH₂ | 1.70, m | 29.0, CH₂ | 1.71, m |
| | 5b | | 1.51, m | | 1.56, m |
| | 6 | 73.5, CH | 5.04, s | 73.3, CH | 5.07, s |
| | OH | | 6.10, br s | | |
| | NH | | 7.23, br s | | 7.23, br s |
| Abu | 1 | 163.0, C | | [symbol] | |
| | 2 | 129.9, C | | [symbol] | |
| | 3 | 131.6, CH | 6.51, q (7.0) | 131.6, CH | 6.51, q (7.1) |
| | 4 | 12.9, CH₃ | 1.49, d (7.0) | 12.9, CH₃ | 1.49, d (7.1) |
| | NH | | 9.20, br s | | |
| Thr | 1 | [symbol] | | [symbol] | |
| | 2 | 55.2, CH | 4.65, m | 54.8, CH | 4.65, m |
| | 3 | 71.4, CH | 5.54, br s | 71.2, CH | 5.54, br s |
| | 4 | 17.6, CH₃ | 1.23, d (6.3) | 17.4, CH3 | 1.23, d (6.4) |
| | NH | | 7.80, br 2 | | 7.70, br s |
| Val | 1 | 171.7, C | | [symbol] | |
| | 2 | 56.5, CH | 4.47, m | 56.3, CH | 4.46, m |
| | 3 | 30.4, CH | 2.09, m | 30.3, CH | 2.09, m |
| | 4 | 19.0, CH₃ | 0.89, d (6.3) | 18.8, CH₃ | 0.88, d (6.8) |
| | 5 | 17.5, CH₃ | 0.82, d (6.7) | 17.3, CH₃ | 0.82, d (6.8) |
| | NH | | 8.13 br s | | 8.18, br s |
| 2-O—CH₃ | 1 | 168.9, C | | [symbol] | |
| Glyceric Acid | 2 | 80.0, CH | 3.98, dd (7.3, 3.4) | 79.8, CH | 3.98, dd (7.3, 3.4) |
| | 3a | 66.0, CH₂ | 3.90, dd (−11.1, 3.4) | 65.8, CH₂ | 3.89, dd (−10.9, 3.4) |
| | 3b | | 3.74, dd (−11.1, 7.3) | | 3.73, dd (−10.9, 7.3) |
| | OCH₃ | 57.4, CH₃ | 3.32^{g} | 57.0, CH₃ | 3.33^{g} |

^{a}Deduced from HSQC and HMBC, 600 MHz.
^{b}600 MHz.
^{c}Refers to symplostatin 6 (2).
^{d}Refers to symplostatin 9 (5).
^{e}Not determined, predicted to have comparable chemical shifts based on highly homologous structures.
^{f}No correlation observed from HMBC.
^{g}Overlapping with residual water. [symbol]
indicates data missing or illegible when filed

TABLE 3
NMR data of symplostatin 7 (3) and symplostatin 10 (6) in DMSO-d₆.

| | C/H | 3 | | 6 | |
|---|---|---|---|---|---|
| Unit | no | δ_{C}^{a} | δ_{H} (J in Hz)^{b} | δ_{C}^{a} | δ_{H} (J in Hz)^{b} |
| Ile | 1 | [symbol] | | [symbol] | |
| | 2 | 54.1, CH | 4.88, br d | 53.9, CH | 4.88, m |
| | 3 | 37.0, CH | 1.88, m | 36.9, CH | 1.86, m |
| | 4a | 26.0, CH₂ | 1.30, m | 25.8, CH₃ | 1.29, m |
| | 4b | | 1.12, m | | 1.12, m |
| | 5 | 11.3, CH₃ | 0.92, t (7.2) | 11.9, CH₃ | 0.91, t (7.4) |
| | 6 | 14.4, CH₃ | 0.71, d (6.7) | 14.2, CH₃ | 0.70, d (6.8) |
| | NH | | 7.44, br s | | 7.38, br s |
| N—Me-Phe^{b}/ | 1 | [symbol] | | [symbol] | |
| N—Me-Tyr^{c} | 2 | 60.3, CH | 5.01, br d | 60.6, CH | 4.90, m |
| | 3a | 33.6, CH₂ | 3.24, br d (−13.4) | 32.5, CH₂ | 3.11, d (−14.2) |
| | 3b | | 2.85, m | | 2.70, dd (−14.2, 11.8) |
| | 4 | [symbol] | | [symbol] | |
| | 5/9 | 129.5, CH | 7.24, d (7.6) | 130.2, CH | 6.99, d (8.3) |
| | 6/8 | 128.4, CH | 7.40, m | 115.1, CH | 6.76, d (8.3) |
| | 7 | 126.6, CH | 7.31, m | | |
| | N—Me | 30.2, CH₃ | 2.79, s | 30.0, CH₃ | 2.74, s |
| | OH | | | | 9.31, br s |
| Phe | 1 | [symbol] | | [symbol] | |
| | 2 | 49.9, CH | 4.72, m | 49.9, CH | 4.72, dd (11.4, 4.8) |
| | 3a | 35.0, CH₂ | 2.85, m | 35.0, CH₂ | 2.87, (−4.3, 12.3) |
| | 3b | | 1.69, m | | 1.79, m |
| | 4 | [symbol] | | [symbol] | |
| | 5/9 | 129.2, CH | 6.83, d (7.4) | 129.2, CH | 6.83, d (7.2) |
| | 6 | 127.6, CH | 7.19, m | 127.6, CH | 7.19, m |
| | 7 | 126.1, CH | 7.16, m | 126.0, CH | 7.14, m |
| Ahp | 2 | [symbol] | | [symbol] | |
| | 3 | 47.9, CH | 3.77, m | 47.9, CH | 3.77, m |
| | 4a | 21.8, CH₂ | 2.39, m | 21.8, CH₂ | 2.39, m |
| | 4b | | 1.57, m | | 1.57, m |
| | 5a | 29.1, CH₂ | 1.71, m | 29.1, CH₂ | 1.71, m |
| | 5b | | 1.55, m | | 1.55, m |
| | 6 | 73.5, CH | 5.06, s | 73.5, CH | 5.06, br s |
| | OH | | 6.04, s | | 6.03, s |
| | NH | | 7.35, br s | | 7.35, br s |
| Abu | 1 | [symbol] | | [symbol] | |
| | 2 | [symbol] | | [symbol] | |
| | 3 | 131.4, CH | 6.48, q (7.1) | 131.4, CH | 6.48, q (7.2) |
| | 4 | 12.8, CH₃ | 1.46, d (7.1) | 12.8, CH₃ | 1.46, d (7.2) |
| | NH | | 9.22, br s | | 9.22, br s |
| Thr | 1 | [symbol] | | [symbol] | |
| | 2 | 55.0, CH | 4.68, m | 55.0, CH | 4.68, m |
| | 3 | 71.6, CH | 5.52, br s | 71.6, CH | 5.52, br s |
| | 4 | 17.5, CH₃ | 1.21, d (6.5) | 17.5, CH₃ | 1.21, d (6.3) |
| | NH | | 8.24, br s | | 8.19, br s |
| Ile | 1 | [symbol] | | [symbol] | |
| | 2 | 55.8, CH | 4.48, m | 55.8, CH | 4.48, m |
| | 3 | 36.9, CH | 1.86, m | 36.9, CH | 1.85, m |
| | 4a | 23.7, CH₂ | 1.43, m | 23.7, CH₂ | 1.43, m |
| | 4b | | 1.06, m | | 1.06, m |
| | 5 | 10.6, CH₃ | 0.80, t (7.4) | 10.6, CH₃ | 0.80, t (7.4) |
| | 6 | 15.0, CH₃ | 0.85, d (6.5) | 15.0, CH₃ | 0.85, d (6.8) |
| | NH | | 7.75, br s | | 7.73, br s |
| 2-O—CH₃ | 1 | [symbol] | | [symbol] | |
| Glyceric Acid | 2 | 79.9, CH | 3.96, dd (7.5, 3.4) | 79.9. CH | 3.96, dd (7.3, 3.2) |
| | 3a | 66.1, CH₂ | 3.88, dd (−10.8, 3.4) | 66.1, CH₂ | 3.88, dd (−10.7, 3.2) |
| | 3b | | 3.72, dd (−10.8, 7.5) | | 3.72, dd (−10.7, 7.3) |
| | OCH₃ | 57.1, CH₃ | 3.31^{g} | 57.1, CH₃ | 3.31^{g} |

^{a}Decuced from HSQC and HMBC, 600 MHz.
^{b}600 MHz.
^{c}Refers to symplostatin 7 (3).
^{d}Refers to symplostatin 10 (6).
^{e}Not determined, predicted to have comparable chemical shifts based on highly homologus structures.
^{f}No correlation observed from HMBC.
^{g}Overlapping with residual water. [symbol]
indicates data missing or illegible when filed

We claim:

1. A compound of formula (I), 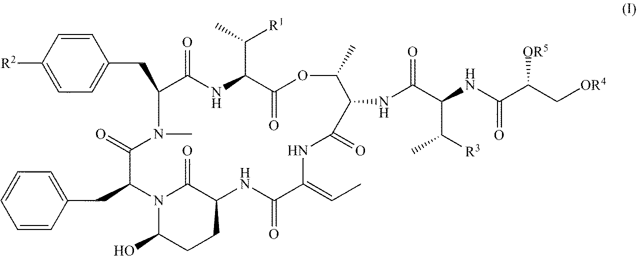
wherein:
R¹ is alkyl;
R² is H or hydroxyl;
R³ is alkyl or hydroxyl;
R⁴ is SO₃M⁺, wherein M⁺ is Na, K, or Li; and
R⁵ is H or alkyl.

2. The compound of claim 1 that is symplostatin 5 (1), symplostatin 6 (2), symplostatin 7 (3), symplostatin 8 (4), symplostatin 9 (5), or symplostatin 10 (6).

3. An isolated compound selected from the group of symplostatin 5 (1), symplostatin 6 (2), symplostatin 7 (3), symplostatin 8 (4), symplostatin 9 (5), or symplostatin 10 (6).

4. A pharmaceutical composition comprising a compound of Formula I and a pharmaceutically acceptable carrier or diluent, wherein: 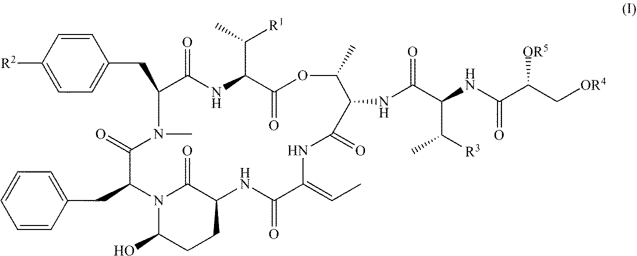
R¹ is alkyl;
R² is H or hydroxyl;
R³ is alkyl or hydroxyl;
R⁴ is H or SO₃M⁺, wherein M⁺ is Na, K, or Li; and
R⁵ is H or alkyl.

5. The composition of claim 4, further comprising an additional therapeutic agent.

6. A method of treating a protease elastase related disease or disorder in a subject, wherein the method comprises administering to the subject an effective amount of a compound of Formula I, wherein: 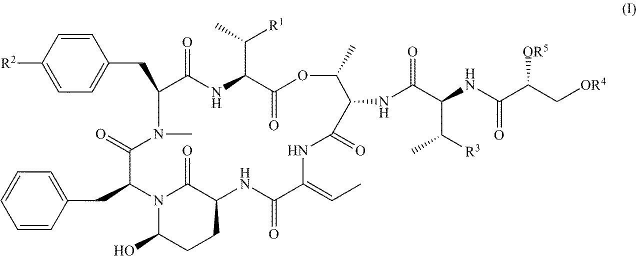
R¹ is alkyl;
R² is H or hydroxyl;
R³ is alkyl or hydroxyl;
R⁴ is H or SO₃M⁺, wherein M⁺ is Na, K, or Li; and
R⁵ is H or alkyl.

7. The method of claim 6, wherein the disease or disorder is chronic obstructive pulmonary disease (COPD), allergic inflammation, asthma, cystic fibrosis or cancer.

8. The method of claim 6, wherein the compound is administered orally.

9. The method of claim 6, wherein the compound is administered topically.

10. A method of inhibiting elastase activity in a subject (e.g., a subject identified as being in need of such treatment), wherein the method comprises administering to the subject an effective amount of a compound of Formula I, wherein: 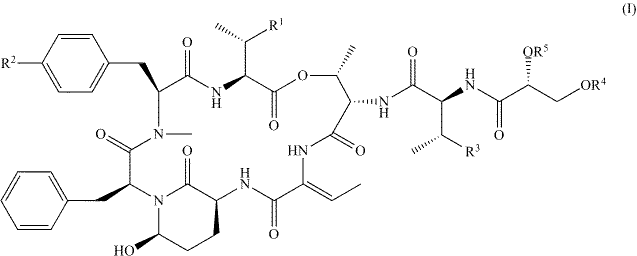
R¹ is alkyl;
R² is H or hydroxyl;
R³ is alkyl or hydroxyl;
R⁴ is H or SO₃M⁺, wherein M⁺ is Na, K, or Li; and
R⁵ is H or alkyl.

11. A method of reducing soluble ICAM-1 production in a subject, wherein the method comprises administering to the subject an effective amount of a compound of Formula I, wherein: 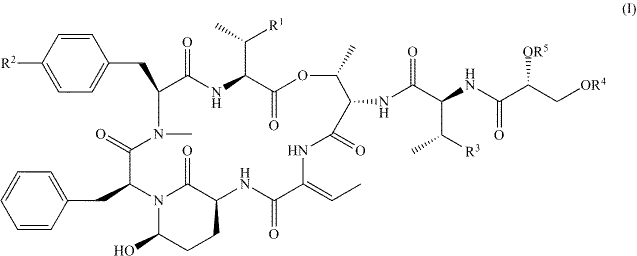
R¹ is alkyl;
R² is H or hydroxyl;
R³ is alkyl or hydroxyl;
R⁴ is H or SO₃M⁺, wherein M⁺ is Na, K, or Li; and
R⁵ is H or alkyl.

12. A composition for topical application to a subject comprising a compound of Formula I and a pharmaceutically acceptable carrier or diluent, wherein: 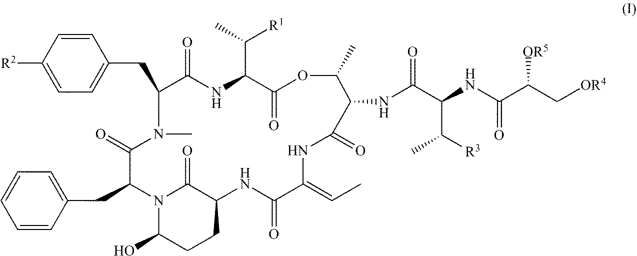
R¹ is alkyl;
R² is H or hydroxyl;
R³ is alkyl or hydroxyl;
R⁴ is H or SO₃M⁺, wherein M⁺ is Na, K, or Li; and
R⁵ is H or alkyl.

13. A compound of claim 1, wherein R⁴ is SO₃M⁺ and M⁺ is Na.

14. Compound of claim 1, wherein R1 is alkyl, R3 is alkyl, R⁴ is SO₃M⁺ and M⁺ is Na.

15. A compound of formula (I 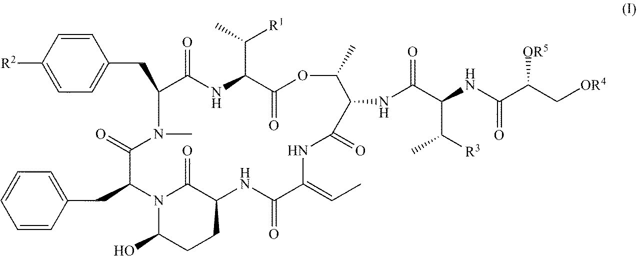
wherein:
R¹ is alkyl;
R² is H or hydroxyl;
R is alkyl or hydroxyl;
R⁴ is H or SO₃M⁻, wherein M⁺ is Na, K, or Li; and
R⁵ is H or alkyl;
with the proviso that when R¹ is alkyl, R² is H, R³ is alkyl, and R⁵ is alkyl, then R⁴ is not H.
